(19) United States
(12) Patent Application Publication
(10) Pub. No.: US 2005/0171455 A1
(43) Pub. Date: Aug. 4, 2005

(54) Pap sphere cervical and endocervical cell home collection device

(76) Inventor: Lorri Neilson Turner, Big Piney, WY (US)

Correspondence Address: Lorri N. Turner, 915 Piney Drive, P.O. Box 1074, Big Piney, WY, 83113-1074, US

(21) Appl. No.: 11/027,608

(22) Filed: Jan. 3, 2005

Related U.S. Application Data
(60) Provisional application No. 60/541,587, filed on Feb. 3, 2004.

(51) Int. Cl. A61B 10/00

(52) U.S. Cl. 600/569; 600/562

(57) ABSTRACT

A cervical and endocervical cell exfoliation and collection device to be used by the female client at home for specimen collection. This device is circular or oval, suitable flexible material, with a flexible ring rim attached to a flexible, thin dome embedded with micro brush fibers. The brush surface may be elevated in one area to assure contact at the cervical os or opening, thus increasing the probability of obtaining endocervical cells. This device will fit behind the cervix where the ring rim will hold it in place and isolate the cervical area inside the dome. The device exfoliates surface cells obtaining them for specimen and the nonporous material collects naturally sloughed cells throughout the sampling period. The nonporous material bathes the cells in the natural mucous secretions and prevents dehydration of the collected cells until the device can be removed, placed in a suitable solution and sent for pathologist review.

Applicant claims the benefit of earlier U.S. provisional patent application No. 60/541,587 Pap Sphere Cervical and Endocervical Cell Home Collection Device Feb. 3, 2004

### CROSS REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE OR A COMPUTER PROGRAM LISTING COMPACT DISC APPENDIX

Not Applicable.

### BACKGROUND OF INVENTION

This invention is a device intended for women to use in the privacy of their own home for the collection of cervical and endocervical cells for pathological and other diagnostic screening. This device relates to an intravaginal device for exfoliation and collection of cells from the cervix and endocervix for diagnostic testing.

The Papanicolaou test is currently the gold standard screening test for cervical cancer. The methods presently available include varied specimen collection and transfer methods all incorporating a physician exam to obtain a specimen using a brush or spatula. This device would enable women to do the exam at their convenience, in the privacy of their own home and with minimal discomfort.

A thorough patent search shows many innovations relating to making the collection of the cervical cells for Pap test more convenient. Most of these devices are similar to a tampon.

The U.K. patent No. 1,455,107 (Denson, Nov. 10, 1976) employs a semi rigid tampon device covered with removable film material. This device is inserted vaginally until it contacts the cervix. The semi rigid nature of this device makes it uncomfortable to wear for any length of time, thus limiting the amount of collection time and number of cells collected. The device design does not ensure contact with the os, or opening of the cervix, where the transitional zone lies. The transitional zone is the site of origin for most cervical cancers. Thus cell collection could occur without any endocervical cells available for pathology review. Any tampon type device containing porous material may become a reservoir for bacteria thus increasing the likelihood of the patient contracting toxic shock, a potentially life-threatening infection.

Many other tampon like devices have been patented, but none are in commercial use do to the afore mentioned drawbacks of short collection time, use of a porous material increasing the likelihood of infection, discomfort and lack of assurance of contacting the transitional zone for cell collection.

Many other patents exist also for other types of self-collection devices for cervical cell sampling. U.S. Pat. No. 4,628,941 (Kasasky, Dec. 16, 1986) utilizes a plunger like tip to push against the cervix and collect the sample. Other similar devices; U.S. Pat. No. 2,844,150 (Draghi, Jul. 22, 1958), U.S. Pat. No. 2,847,000 (Nieburgs, Aug. 12, 1958), U.S. Pat. No. 6,155,990 (Fournier, Dec. 5, 2000), U.S. Pat. No. 6,352,513 (Anderson et. al., Mar. 5, 2002), all incorporate some type of plunger or tube. All of these type devices share shortened specimen collection time, possible absence of contact at the transitional zone for collection of endocervical cells, uncomfortable insertion for the patient and possibility of cells drying out before contacting fixative medium due to the porous nature of the collection device.

One device that is similar in idea to the proposed design incorporates a device made of high density, non-absorbent non-fibrous polyurethane foam. U.S. Pat. No. 5,231,992 (Leon, Aug. 3, 1993) describes a disc shaped device made of foam with an inner well of cell moistening gel covered with a stiff, porous membrane. The device is inserted in its entire length and width, with an inner rounded edge to ease insertion. This device sits adjacent to the cervix where cells and fluid are collected on the membrane. The device is discarded through the vagina and placed into fixative solution. The intent is for this design to be used by a primary health care provider during the exam.

This design, though an improvement on previous tampon and plunger or tube designs, has several drawbacks as well. The sponge nature of the main body of the device while softer is also slightly abrasive to the walls of the vagina and may cause irritation leading to infection. In addition, the sponge is soft enough that it may double over inside the canal and block off the membrane-collecting device. A separate concern for safety is though the sponge is soft, the inner membrane is thick and stiff and the inventor states in his patent that precautions may have to be taken to prevent breakage of the membrane in the vaginal canal. The 24-hour duration of wearing the device lengthens the collection period but may be so long as to cause irritation and infection. The device fits inside the vaginal canal and creates a barrier, but does not extend behind the cervix to assure contact with the cervical surface, the cells collected would be sloughed cells but no scraped cells would be assured, thus only older cells may be represented. Finally, the membrane itself is porous and the moist gel may become contaminated and combined with the collection of fluid and cells on the membrane may create the same environment as a tampon leading to toxic shock.

Therefore, it is the intent of this invention to provide a cervical and endocervical cell collection device that not only contacts but partially encapsulates the cervix to assure contact at the transitional zone, that is non-porous to limit occurrence of toxic shock and other infections, that folds for comfort of insertion and may be worn comfortably and safely for a suitable time period, for example 1-8 hours, provides a tight fit surrounding the cervix that will not allow the device to shift or double over inside the vaginal cavity, is made of a smooth, suitable, nonporous material that decreases the likelihood of irritating the vaginal walls, and that provides a mildly abrasive brush type collection allowing for cells to be exfoliated and sloughed off to give a more varied cell sample for diagnosis.

### BRIEF SUMMARY OF INVENTION

The invention incorporates a cell collection device for collecting cervical and endocervical cells by exfoliation and natural cell sloughing, at home, with a flexible material type ring rim that can easily fold in half and then will revert to its original circular shape in the vaginal canal, a thin, silicone or other suitable material dome connected to the rim in original molded design, an area of micro bristles embedded into the silicone dome's inner surface comprising a brush like surface to the inner portion of the dome with or without elevated bristles toward the center to assure contact with the cervical os. The smooth outer surface in contact with the vaginal mucosa will limit irritation.

The design can be manufactured in varying sizes, for instance, small 1½ inch diameter for young, nulliparous and elderly women, 2 inch diameter for childbearing age women of average stature, 2½ inch diameter for large and multiparous women allowing for a more comfortable and efficient fit of the device.

The device can be contained in a home kit with a container of fixative solution and gloves allowing home use. This device should fit comfortably in the vaginal canal without causing irritation or infection for approximately 1-8 hours ensuring varied cell collection. The device can also be adapted to obtain cultures for infectious disease diagnosis, hormone identifiers and other diagnostic tests if contained in a kit with the proper medium.

### BRIEF DESCRIPTION OF THE SEVERAL VIEWS OF THE DRAWING

FIG. 1: Referring to FIG. 1, Pap Sphere, a cervical and endocervical cell collector device in a frontal, inner view, viewing the device inside the dome.

FIG. 2: FIG. 2 shows a cross-sectional view of the inner surface of the Pap Sphere device with the flexible ring rim 1, inner dome with micro brush bristles 2, and elevated bristle areas 3.

FIG. 3: FIG. 3 represents the flexible nature of the ring rim. The flexible ring rim 1, will fold in half lengthwise between the thumb and forefinger with the micro brush surfaces 2 and 3, kept toward the inside. Once placed in the vaginal canal and pushed toward the cervix the device will unfold into its circular shape 4 and 5.

FIG. 4: FIG. 4 shows the Pap Sphere device in place in an anatomical coronal view of a woman's body. The uterus 6, extending downward into the cervix 7. The anatomical locations for the bladder 8 and the vagina 9 are indicated for viewing purposes. The Pap Sphere device in place covering the cervical surface is represented 10.

### DETAILED DESCRIPTION OF THE INVENTION

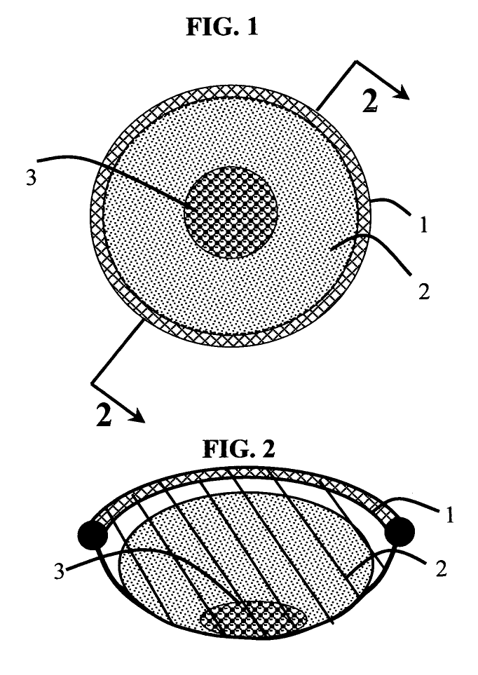

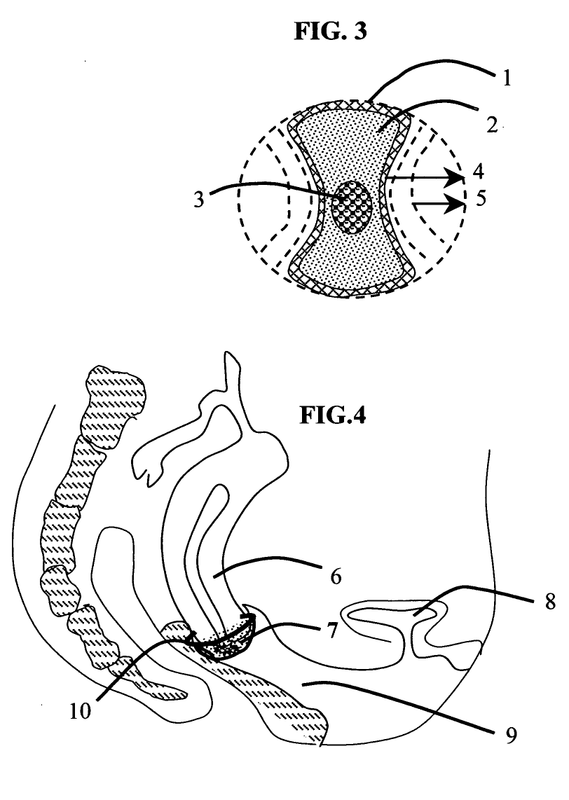

Referring to FIG. 1, the Pap Sphere, cervical and endocervical cell collection device is composed of a flexible ring rim 1, molded to a dome shaped collection device 2, and made of suitable material, for example silicone. The rim 1, may be made in varying sizes to fit different body types. The rim 1, should be circular or oval and flexible enough to be compressed FIG. 3, to fit into the vaginal opening in a vertical, compressed position. Once inserted it would return to its original circular shape due to the decreased tension and elasticity of the vaginal walls and could be eased upward to enclose the cervix.

The ring rim FIG. 4, reference 1, would fit behind the cervix and be held in place by tension against the vaginal walls FIG. 4. The dome would fit over the cervical surface FIG. 4, effectively encapsulating the target cell area.

Referring to FIG. 1 a full inner view and FIG. 2 a cross-sectional view, the dome reference 2, would be embedded with small fibers or micro bristles extending from and covering the inner surface. The dome 2, could also contain an area with elevated or longer fibers or micro bristles 3. The elevated bristles 3, would contact the cervical os or opening and exfoliate cells from the transitional zone. The fibers or micro bristles covering the rest of the inside surface of the dome 2, would contact the face of the cervix, exfoliating and collecting cells from the entire outer cervical area. The outer surface of the dome would not contain fibers or micro bristles and would therefore remain a smooth silicone surface, which should minimize discomfort and irritation.

According to the invention, FIG. 3 demonstrates the flexible nature of the ring rim. The flexibility of the rim 1, should allow the device to be compressed between the fingers and thumb for easier insertion into the vaginal canal opening. The flexibility should provide enough tension for the device to unfold in the vaginal canal due to decreased pressure and increased elasticity of the vaginal walls, FIG. 3, reference 4, and return to its original circular or oval shape 5, as it is eased into place. Proper placement would be verified by finger insertion to feel the device in place with the firm surface of the cervix isolated in the dome. The device would fit snugly until it is dislodged by finger insertion and extracted to be placed in the proper medium for testing.

According to the invention, the embedded bristles FIG. 1, reference 2 and 3, would contact the cervical surface. Normal movement incurred with active daily life would create friction of the cervical surface with the fibers or micro bristles inside the dome causing exfoliation of cervical and endocervical cells for collection. In addition, normal cell sloughing of aging cells would take place during the collection time period adding to the variety of the cells sampled on the device for evaluation. The nonporous nature of the invention would also collect cervical mucous and normal secretions, which would bathe the cells preventing dehydration until the device is extracted.

In the preferred test protocol, the device is used for exfoliation and collection of endocervical and cervical cells for detection of precancerous and cancerous conditions of the cervix. The device, according to the invention may also be used to test for infectious diseases, hormone identifiers and any other disease entity that manifests itself with cells or antibodies that can be collected or exfoliated by the device.

According to the invention, the device may be used for rapid cell collection, but in the preferred protocol, the device is left in place for approximately one to eight hours to allow for better collection of sloughed cells.

At the end of the collection time period, according to the invention, the female client will remove the device with a gloved finger and place it in the appropriate fixative or suitable solution for preservation of the cells for testing.

Thus, there is provided a simple, convenient method intended for use by a female client at home, for exfoliating and collecting cervical and endocervical cells and fluids for diagnostic purposes.

From the foregoing description of the preferred embodiments of the invention, it will be apparent that many modifications may be made therein. It will be understood, however, that these embodiments of the invention are exemplifications of the invention only and that the invention is not limited thereto. It is to be understood therefore that it is intended in the appended claims to cover all modifications that fall within the true spirit and scope of the invention.

1. An internally worn device for exfoliating and collecting cells and fluids from the endocervical and cervical surfaces of a female client comprising a flexible material ring rim made of suitable material, for example silicone, molded to a dome of similar material embedded on its inner surface with fibers or micro brush bristles made of suitable material, for example nylon, with or without an area of elevation or longer fibers or micro brush bristles, that when in proper position fits behind the cervix and isolates the cervix, os and transitional zone inside the dome with the fibers or micro bristles in contact with the cervical os or opening.

2. The device in claim 1, wherein the cells and fluids collected can be used for pathological or laboratory screening to screen for cervical conditions such as abnormal or atypical, precancerous or cancerous endocervical, cervical or vaginal cells.

3. The device in claim 1, wherein the cells collected can be used for pathological or laboratory screening to aid diagnosis of infectious diseases when placed in suitable preservation solution or culture medium.

4. The device in claim 1, wherein the cells collected can be used by laboratory and pathology personnel to identify DNA markers when placed in suitable preservation or culture medium.

5. The device in claim 1, wherein the cells collected can be used by laboratory and pathology personnel to identify hormone identifiers when placed in suitable preserve-ation or culture medium.
